(19) United States
(12) Patent Application Publication
(10) Pub. No.: US 2013/0123872 A1
(43) Pub. Date: May 16, 2013

(54) LEADLESS IMPLANTABLE MEDICAL DEVICE WITH DUAL CHAMBER SENSING FUNCTIONALITY

(71) Applicant: PACESETTER, INC., Sylmar, CA (US)

(72) Inventors: Gene A. Bornzin, Simi Valley, CA (US); John W. Poore, South Pasadena, CA (US)

(73) Assignee: PACESETTER, INC., Sylmar, CA (US)

(21) Appl. No.: 13/669,168

(22) Filed: Nov. 5, 2012

Related U.S. Application Data
(60) Provisional application No. 61/555,421, filed on Nov. 3, 2011.

(51) Int. Cl. A61N 1/365

(52) U.S. Cl. CPC A61N 1/36592
USPC 607/17

(57) ABSTRACT

A leadless implantable medical device (LIMD) is provided with dual chamber sensing functionality, without leads, despite the fact that the entire device is located in one chamber. In one embodiment, the LIMD senses local activity in the right atrium (RA) and local activity in the right ventricle (RV), even though it is entirely located in the RA. The sensing electrodes enable sensing in different chambers of the heart while reducing cross talk interference and thus provide accurate tracking of myocardial contraction in multiple chambers.

### CROSS REFERENCE TO RELATED APPLICATIONS

This application relates to and claims priority benefits from U.S. Provisional Application No. 61/555,472, filed Nov. 3, 2011, entitled “Single Chamber Leadless Implantable Medical Device Having Dual Chamber Sensing with Far Field Signal Rejection,” which is hereby incorporated by reference in its entirety.

### BACKGROUND OF THE INVENTION

Embodiments of the present invention generally relate to leadless implantable medical devices, and more particularly to leadless implantable medical devices that afford dual chamber sensing functionality from a position within a single chamber of the heart.

Currently, permanently-implanted pacemakers (PPMs) utilize one or more electrically-conductive leads (which traverse blood vessels and heart chambers) in order to connect a canister with electronics and a power source (the can) to electrodes affixed to the heart for the purpose of electrically exciting cardiac tissue (pacing) and measuring myocardial electrical activity (sensing). These leads may experience certain limitations, such as incidences of venous stenosis or thrombosis, device-related endocarditis, lead perforation of the tricuspid valve and concomitant tricuspid stenosis; and lacerations of the right atrium, superior vena cava, and innominate vein or pulmonary embolization of electrode fragments during lead extraction.

A small sized PPM device has been proposed with leads permanently projecting through the tricuspid valve that mitigate the aforementioned complications. This PPM is a reduced-size device, termed a leadless pacemaker (LLPM), that is characterized by the following features: electrodes are affixed directly to the CAN of the device; the entire device is attached to the heart; and the LLPM is capable of pacing and sensing in the chamber of the heart where it is implanted.

LLPM devices, that have been proposed thus far, offer limited functional capability. These LLPM devices are able only to sense local activity in one chamber and deliver pacing pulses in that same chamber. For example, an LLPM device that is located in the right atrium would be limited to offering AAI mode functionality. An AAI mode LLPM can only sense local activity in the right atrium, pace in the right atrium and inhibit pacing function when an intrinsic local event is detected in the right atrium within a preset time limit. Similarly, an LLPM device that is located in the right ventricle would be limited to offering VVI mode functionality. A VVI mode LLPM can only sense local activity in the right ventricle, pace in the right ventricle and inhibit pacing function when an intrinsic event is detected in the right ventricle within a preset time limit.

Cardiac pacemaker lead systems fulfill two functions. The first function is to provide an electrical conduit by which a pacemaker output pulse is delivered to stimulate the local tissue adjacent to the distal tip of the lead. The second function is to sense local, intrinsic cardiac electrical activity that takes place adjacent to the distal tip of the lead.

With the introduction of leadless pacemaker devices, one of the problems is their inability during sensing to suppress or attenuate the voltage levels of far-field electrical signals that are sensed. These far-field signals are generated by depolarizations of body tissue in areas remote from the local sensing site and are manifested as propagated voltage potential wave fronts carried to and incident upon the local sensing site. A far-field signal may comprise an intrinsic or paced signal originating from a chamber of the heart other than the one in which the sensing electrodes are located. The sensing electrode(s) detect or sense the voltages of these far-field signals and interpret them as depolarization events taking place in the local tissue when such polarizations are above the threshold sensing voltage of the LLPM. When far-field signal voltages greater than the threshold voltage are applied to the sensing circuitry of the LLPM, activation of certain pacing schemes or therapies can be erroneously triggered.

With the development of multi-chamber LLPM systems, accurate sensing of cardiac signals has become even more important. The management, suppression and/or elimination of far-field signals is very desirable to allow appropriate device algorithms to function without being confused by the undesirable far-field signals that are sensed as cross-talk when using unipolar electrodes. Otherwise, cross-talk may cause sensing ambiguity.

For a sensing electrode implanted in the right atrium, the right ventricular R-wave comprises a far-field signal whose amplitude can easily dominate and overshadow the smaller P-wave signal sought to be sensed. Thus, the discrimination of i) P-waves from the higher energy QRS complexes and ii) the R-wave spikes continues to present a formidable challenge.

It is known that in a bipolar pacing and sensing lead, the reference electrode (or anode), typically in the form of an electrically conductive ring disposed proximally of the tip cathode electrode, should have a large active surface area compared to that of the cathode. The objects of such an areal relationship are to reduce the current density in the region surrounding the anode so as to prevent needless or unwanted stimulation of body tissue around the anode when a stimulation pulse is generated between the cathode and anode, and to minimize creation of two focal pacing sites, one at the cathode and one at the anode which could promote arrhythmia. Typically, the total surface area of the anode is selected so as to be about two times to about six times that of the cathode.

Despite the advances in the field, there remains a need for a bipolar, sensing configuration that sufficiently attenuate far-field signals while at the same time providing clinically acceptable near-field signals for reliable sensing. Moreover, the need exists for such a system that can be located in association with any chamber of the heart, and that can sufficiently attenuate far-field signals.

### SUMMARY OF THE INVENTION

In accordance with one embodiment, a leadless implantable medical device (LIMD) is provided with dual chamber sensing functionality, without leads, despite the fact that the entire device is located in one chamber. In one embodiment, the LIMD senses local activity in the right atrium (RA) and local activity in the right ventricle (RV), even though it is entirely located in the RA. The sensing electrodes enable sensing in different chambers of the heart while reducing cross talk interference and thus provide accurate tracking of myocardial contraction in multiple chambers.

The LIMD comprises a housing configured to be implanted entirely within a single local chamber of the heart. The local chamber has local wall tissue that constitutes part of a conduction network of the local chamber. A controller within the housing causes stimulus pulses to be delivered. A sensing circuit performs sensing. An active fixation member is coupled to the housing and is configured to be secured to a septum that separates the local chamber from an adjacent chamber. The adjacent chamber has distal wall tissue, with respect to the local chamber that constitutes part of a conduction network of the adjacent chamber. The active fixation member has a distal segment configured to extend at least partially through the septum to a distal sensing site proximate to the distal wall tissue within the conduction network of the adjacent chamber. An electrode pair has first and second active electrode areas coupled to the sensing circuit. The first and second electrode areas are positioned such that, when the LIMD is implanted, the electrode pair is electrically coupled to the conduction network of the adjacent chamber. The sensing circuit detecting, as near field signals, voltages originating within the conduction network of the adjacent chamber and sensed between the first and second active electrode areas. The sensing circuit rejecting, as far field signals, voltages originating within the conduction network of the local chamber and sensed by the first and second active electrode areas.

Optionally, the sensing circuit and electrode pair are coupled to operate in a bipolar sensing configuration such that the sensing circuit measures a voltage potential difference between the first and second active electrode areas. Optionally, the active fixation member is helical in shape, the first and second active electrode areas being located on separate turns of the active fixation member and at a common distance from the base. Optionally, the electrode pair is provided on the active fixation member within the distal segment thereof, such that when the active fixation member is installed, the electrode pair are located at or near a surface of the distal wall tissue.

Optionally, the LIMD further comprises a pin that extends from the base of the housing, the pin having a distal end with the active electrode areas provided at the distal end of the pin. The LIMD may have a second electrode pair which has third and fourth electrodes that are provided on the base of the housing. The third and fourth active electrode areas are coupled to the sensing circuit and positioned such that the second electrode pair is electrically coupled to the conduction network of the local chamber. The sensing circuit detecting, as near field signals, voltages originating within the conduction network of the local chamber and sensed by the third and fourth electrodes. The sensing circuit rejecting, as far field signals, voltages originating within the conduction network of the adjacent chamber and sensed by the third and fourth electrodes.

The active fixation member includes a proximal segment configured to extend into the septum to the local sensing site. The LIMD may be further comprised of a second electrode pair provided on the active fixation member in the proximal segment to be electrically coupled to the conduction network of the local chamber. The sensing circuit may detect, as near field signals, voltages originating within the conduction network of the local chamber. The sensing circuit rejects, as far field signals, voltages originating within the conduction network of the adjacent chamber.

The active fixation member includes first and second electrode pairs that are located within proximal and distal segments of the active fixation member, respectively, the electrodes in the proximal segment being positioned to be electrically coupled to the conduction network of the local chamber.

The first and second electrodes are separated by an inter-electrode spacing that is sufficient such that as depolarization occurs along the distal wall tissue and near field electrical activity moves across the first and second electrodes. An associated voltage potential is created between the first and second electrodes, the voltage potential being detected by the sensing circuit as a near field signal.

The first and second electrodes are separated by an inter-electrode spacing such that as far field electrical activity traverses the first and second electrodes, a common mode signal experienced between the first and second electrodes, the common mode signal being rejected by the sensing circuit.

A method for providing a leadless implantable medical device (LIMD), comprised of a housing configured to be implanted entirely within a single local chamber of the heart. The local chamber has local wall tissue that constitutes part of a conduction network of the local chamber and configures a controller within the housing to cause stimulus pulses to be delivered. The method includes configuring a sensing circuit to perform sensing and coupling an active fixation member to the housing. The active fixation member is configured to be secured to a septum that separates the local chamber from an adjacent chamber. The adjacent chamber has distal wall tissue, with respect to the local chamber that constitutes part of a conduction network of the adjacent chamber. The active fixation member has a distal segment configured to extend at least partially through the septum to a distal sensing site proximate to the distal wall tissue within the conduction network of the adjacent chamber. The active fixation member provides an electrode pair having first and second electrodes coupled to the sensing circuit. The first and second electrodes are positioned such that the electrode pair is electrically coupled to the conduction network of the adjacent chamber. The sensing circuit detects, as near field signals, voltages originating within the conduction network of the adjacent chamber and sensed between the first and second electrodes. The sensing circuit rejects, as far field signals, voltages originating within the conduction network of the local chamber and sensed between the first and second electrodes.

Broadly, embodiments provide, a LIMD that provides bipolar sensing utilizing a range of locations and configurations of active surface areas for each of the anode and cathode electrodes, and a range of inter-electrode spacings between the anode and cathode electrodes which, in combination, afford good discrimination of the sensed near-field signal and a desired ratio of the near-field to far-field signal amplitudes, that is, the signal-to-noise ratio.

The active sensing electrode pair areas described herein when located in the right atrium, afford clinically acceptable distal local event R-wave near Field signal amplitudes while significantly attenuating P-wave far-field signals. In addition, in the case of a ventricular implant, the LIMD provides acceptable P-wave near Field signal amplitudes and mitigates R-wave oversensing and attenuates far-field R-wave signals, without compromising autocapture and morphology discrimination. Further, the inter-electrode spacing is sufficient to inhibit fibrotic encapsulation of the active electrode areas and the consequent formation of a “virtual electrode”. Such inhibition may be further enhanced by incorporating a steroid collar between the active electrode areas of an electrode pair.

### BRIEF DESCRIPTION OF THE DRAWINGS

FIG. 1 illustrates a sectional view of the patient's heart and shows a leadless implantable medical device.

FIG. 2 illustrates a right anterior oblique view representing the interior surface of the right atrium wall.

FIG. 3A illustrates a bottom perspective view of the LIMD of FIG. 1.

FIG. 3B illustrates a bottom plan view of the LIMD of FIG. 1.

FIG. 3C illustrates examples of locations where the LIMD may be implanted.

FIG. 4A illustrates a side view of an end portion of an LIMD in accordance with an embodiment.

FIG. 4B illustrates a distal segment of an active fixation member formed in accordance with an embodiment.

FIG. 5 illustrates an LIMD formed in accordance with an alternative embodiment.

FIG. 6 illustrates a bottom plan view of an LIMD.

FIG. 7 illustrates a block diagram of an exemplary switching circuit that may be used in accordance with an embodiment of the present invention.

FIG. 8 illustrates an exemplary block diagram of the electrical components of an LIMD in accordance with an embodiment.

FIG. 9 illustrates a bottom plan view of an LIMD formed in accordance with an alternative embodiment.

FIG. 10 illustrates a bottom plan view of an LIMD formed in accordance with an alternative embodiment.

### DETAILED DESCRIPTION

Dual-chamber PPMs, operating in the DDD or DDDR mode, are indicated for patients with complete atrioventricular (AV) block, sick sinus syndrome, and paroxysmal AV block. The use of DDD or DDDR mode PPMs in patients with a high degree of AV block is shown to improve subjective metrics of patient life and increase peak velocity and cardiac output, compared to VVIR PPMs. Additionally, another study demonstrates reduced incidence of atrial fibrillation (AF) and increased patient longevity in patients with sick sinus syndrome after the time of DDD or DDDR PPM implant. These significant benefits, accrued to the three previously-described subgroups of implant patients, provide a strong impetus for using DDD or DDDR PPMs in those recipients.

The benefits of conventional DDD or DDDR PPMs are counterbalanced by the increased risk of complications with the additional lead necessary for these PPMs (compared to single-chamber devices). A preferred solution to this dilemma as offered by embodiments herein eliminates the need to use leads by providing an LIMD with DDD or DDDR mode functionality. As a result, patients suffering from various degrees of AV block or sick sinus syndrome may receive dual-chamber pacing therapy without an increased risk of complications (such as lead-associated infections caused by biofilm formation or explant-related difficulties). In particular, decreased incidence of device-related infections may be achieved by a DDD or DDDR mode-capable LIMD as a result of the device body's small surface area (compared to conventional PPMs and leads), which presents a reduced substrate for bacterial or fungal adhesion.

Myocardial contraction results from a change in voltage across the cell membrane (depolarization), which leads to an action potential. Although contraction may happen spontaneously, it is normally in response to an electrical impulse. In normal physiologic behavior, this impulse starts in the sino-atrial (SA) node where a collection of cells are located at the junction of the right atrium and superior vena cava. These specialized cells depolarize spontaneously, and cause a wave of contraction to follow a conduction network along the tissue wall of the atria. Following atrium contraction, the impulse is delayed at the atrio-ventricular (AV) node, located in the septum wall of the right atrium. From here HIS-Purkinje fibers allow rapid conduction of the electrical impulse to propagate along the conduction network formed by the right and left branches in the RV and LV tissue walls, causing almost simultaneous depolarization of both ventricles, approximately 0.2 seconds after the initial impulse has arisen in the sino-atrial node. Depolarization of the myocardial cell membrane causes a large increase in the concentration of calcium within the cell, which in turn causes contraction by a temporary binding between two proteins, actin and myosin. The cardiac action potential is much longer than that of skeletal muscle, and during this time the myocardial cell is unresponsive to further excitation. Hence, in a general sense, the tissue walls of each chamber constitute part of a conduction network of the corresponding chamber.

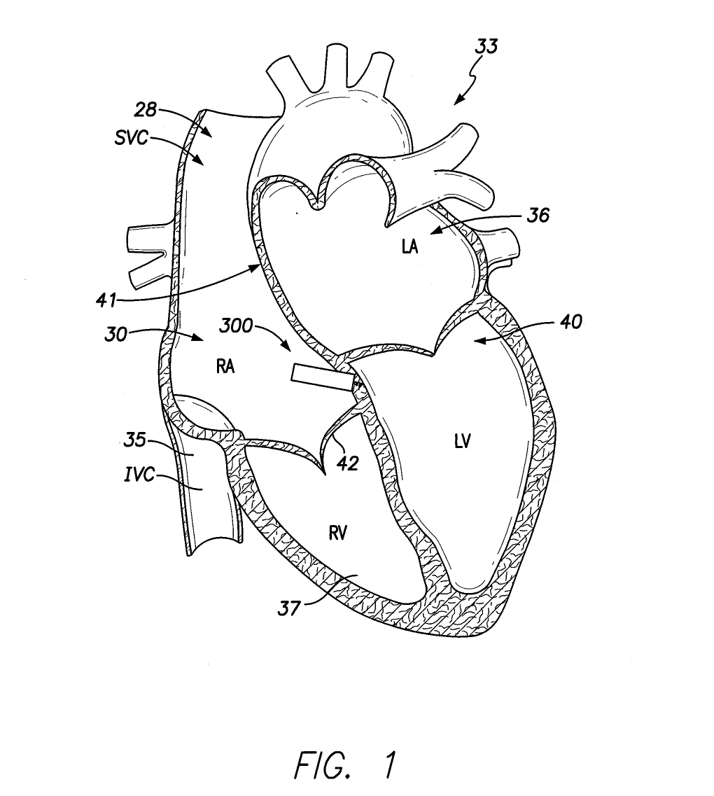

FIG. 1 provides a sectional view of the patient's heart 33 and shows a leadless implantable medical device 300. The leadless implantable medical device 300 has been placed through the superior vena cava 28 into the right atrium 30 of the heart 33. FIG. 1 also shows the inferior vena cava 35, the left atrium 36, the right ventricle 37, the left ventricle 40, the atrial septum 41 that divides the two atria 30, 36, and the tricuspid valves 42 between the right atrium 30 and right ventricle 37. The reader will appreciate that the view of FIG. 1 is simplified and somewhat schematic, but that nevertheless FIG. 1 and the other views included herein will suffice to illustrate adequately the placement and operation of embodiments of the present invention. The term “septum” shall be used throughout to generally refer to any portion of the heart separating two chambers (e.g. RA to LA, RV to LV). The leadless implantable medical device (LIMD) 300 is formed in accordance with an embodiment herein. The LIMD 300 may represent a pacemaker that functions in a DDD or DDDR-mode, a cardiac resynchronization device, a cardioverter, a defibrillator and the like. When in DDD or DDDR-mode, the LIMD 300 may sense in two chambers, pace in two chambers and inhibit pacing in either chamber based on intrinsic events sensed in that chamber or in the other chamber. The LIMD 300 comprises a housing configured to be implanted entirely within a single local chamber of the heart. For example, the LIMD 300 may be implanted entirely and solely within the right atrium or entirely and solely within the right ventricle. Optionally, the LIMD 300 may be implanted entirely and solely within the left atrium or left ventricle through more invasive implant methods.

For convenience, hereafter the chamber in which the LIMD 300 is implanted shall be referred to as the “local” chamber. The local chamber includes a local chamber wall that is physiologically responsive to local activation events originating in the local chamber. The local chamber is at least partially surrounded by local wall tissue that forms or constitutes at least part of a conduction network for the associated chamber. For example, during normal operation, the wall tissue of the right atrium contracts in response to an intrinsic local activation event that originates at the sinoatrial (SA) node and in response to conduction that propagates along the atrial wall tissue. For example, tissue of the right atrium chamber wall in a healthy heart follows a conduction pattern, through depolarization, that originates at the SA node and moves downward about the right atrium until reaching the atria ventricular (AV) node. The conduction pattern moves along the chamber wall as the right atrium wall contracts.

The term “adjacent” chamber shall refer to any chamber separated from the local chamber by tissue (e.g., the RV, LV and LA are adjacent chambers to the RA; the RA and LV are adjacent chambers to the LA; the RA and RV are adjacent to one another; the RV and LV are adjacent to one another, and the LV and LA are adjacent to one another).

The local chamber (e.g., the right atrium) has various tissue of interest, such as a septum, which separate the local chamber from the adjacent chambers (e.g., right ventricle, left atrium, left ventricle). Certain portions or segments of the septum, behave in physiologically different manners. For example, in certain segments of the septum for the right atrium, even during normal healthy operation, the septum wall tissue does not propagate the conduction in the same manner or pattern as in a majority of the wall tissue of the right atrium wall. For example, septum wall tissue in the right atrium, referred to herein as the ventricular vestibule, does not behave physiologically in the same manner as the non-septum atrial wall tissue. Instead, the ventricular vestibule is physiologically coupled to the wall tissue in the right ventricle and in accordance therewith exhibits a conduction pattern that follows the conduction pattern of the right ventricular wall tissue. The ventricular vestibule tissue is one example of a septum segment that partially separates a local chamber (e.g., the right atrium) from an adjacent chamber (e.g., left ventricle), yet is physiologically coupled to conduction in the adjacent chamber (e.g., left ventricle).

In the example of FIG. 1, the LIMD 300 is implanted in an area near different regions of tissue that follow the conductive pattern of different chambers of the heart. Optionally, the LIMD 300 may be implanted such that at least one electrode on the base of the LIMD 300 engages tissue that is part of the conductive network of the one chamber, while at least one other electrode projects from the base into tissue that is part of the conductive network of another chamber. For example, when the LIMD 300 may be implanted within or near the triangle of Koch in an area adjacent the ventricular vestibule. The conductive network of the tissue in the ventricular vestibule follows the conductive pattern of the right ventricle. Therefore, the LIMD 300 may be implanted near the edge of the triangle of Koch such that one or more proximal electrodes, extending from the LIMD 300, are electrically coupled to the conductive network of the right atrium, while one or more other distal electrodes, extend diagonally to become electrically coupled to the conductive network of the right ventricle (e.g., the ventricular vestibule). Optionally, the LIMD 300 may be positioned with the base located against the RA wall above the mitral valve, but with a distal electrode that projects into the septum to ventricular tissue of the right or left ventricle.

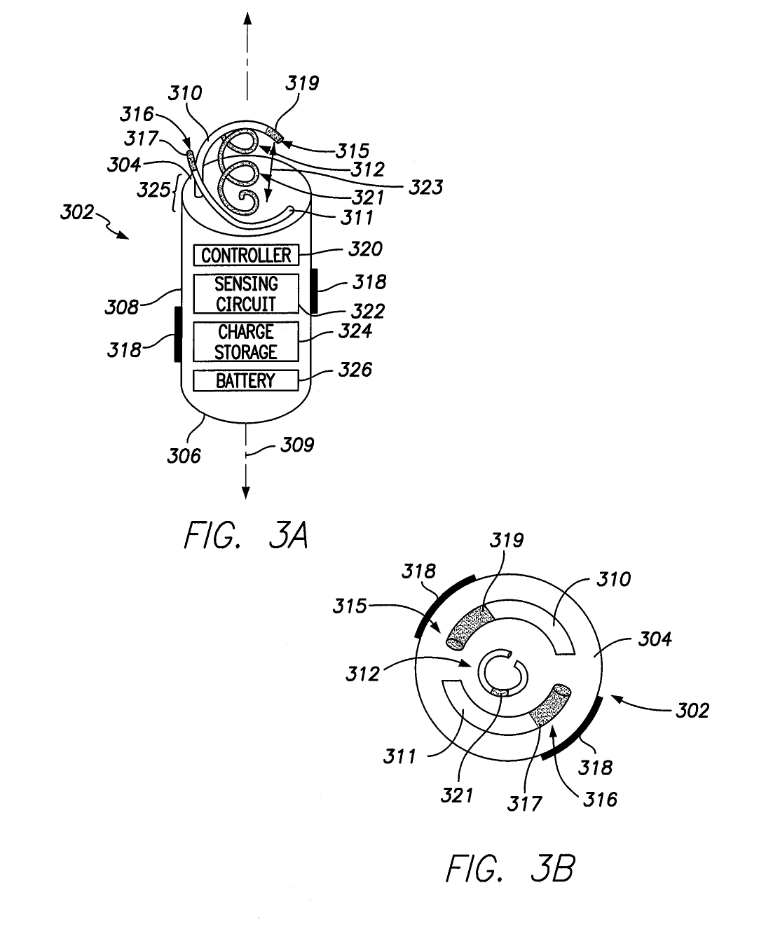

FIGS. 3A and 3B illustrate the LIMD 300 in more detail. FIG. 3A illustrates a bottom perspective view of the LIMD 300 of FIG. 1. FIG. 3B illustrates a bottom plan view of the LIMD 300. The LIMD 300 comprises a housing 302 having a base 304, a distal top end 306, and an intermediate shell 308 extending between the proximal base 304 and the distal top end 306. The shell 308 is elongated and tubular in shape and extends along a longitudinal axis 309.

The base 304 includes one or more electrodes 310-312 securely affixed thereto and projected outward. For example, the electrodes 310 and 311 may be formed as large semi-circular spikes or large gauge wires that wrap only partially about the inner electrode 312. The electrodes 310 and 311 may be located on opposite sides of, and wound in a common direction with, the inner electrode 312. The first or outer electrodes 310, 311 are provided directly on the housing 302 of the LIMD 300 at a first position, namely at or proximate a periphery of the base 304 of the housing. The outer electrodes 310, 311 are positioned near the periphery of the base 304 such that, when the LIMD 300 is implanted in the local chamber (e.g., right atrium), the outer electrodes 310, 311 engage the local chamber wall tissue at tissue of interest for a local activation site that is near the surface of the wall tissue, and that is within the conduction network of the local chamber. The outer electrodes 310, 311 are physically separated or bifurcated from one another and have separate distal outer tips 315, 316. The outer electrodes 310, 311 are electrically joined to one another (i.e., common), but are electrically separated from the inner electrode 312.

The second or inner electrode 312 is also provided directly on the housing 302 of the LIMD 300 at a second position, namely at or proximate to a central portion of the base 304 of the housing. The inner electrode 312 is positioned near the center of the base 304 and is elongated such that, when the LIMD 300 is implanted in the local chamber, the inner electrode 312 extends a majority of the way through the wall tissue (e.g. septum) until reaching tissue of interest near the adjacent chamber wall. The inner electrode 312 is inserted to a depth such that a distal tip thereof is located at tissue of interest for an activation site that is physiologically coupled to wall tissue of the adjacent chamber (e.g. right ventricle). For example, the inner electrode 312 may extend until the distal tip extends at least partially through a septum to a position proximate to a distal wall tissue within the conduction network of the adjacent chamber. Optionally, the inner electrode 312 may be inserted at a desired angle until the distal end enters the ventricular vestibule. By located the distal tip of the inner electrode 312 at an adjacent chamber activation site, the inner electrode 312 initiates contraction at a distal activation site within the conduction network of the adjacent chamber without physically locating the LIMD 300 in the adjacent chamber. The inner and outer electrodes 310-312 may be formed as multiple cathode electrodes that are actively fixated to the myocardium. The outer cathode electrodes 310, 311 may be configured as screws with a large pitch (e.g. length between adjacent turns), large diameter and may have a length that is relatively short, while the inner electrode 312 is configured as a screw with a common or smaller pitch, small diameter and longer length. The screw shape of the outer electrodes 310, 311 is used to firmly adhere them to the cardiac tissue. The outer electrodes 310, 311 may have very little or no insulation material thereon to facilitate a good electrical connection to local wall tissue along the majority or the entire length of the outer electrodes 310, 311 for delivering stimulus pulses and sensing electrical activity in the local chamber where the LIMD 300 is located.

The second or inner electrode 312 is also provided directly on the housing 302 of the LIMD 300 at a second position, namely at or proximate to a central portion of the base 304 of the housing 302. The inner electrode 312 is positioned near the center of the base 304. When the LIMD 300 is implanted in the local chamber, the inner electrode 312 extends a proximal or short way into the wall tissue or septum tissue segment just below the surface of the local wall tissue. The inner electrode 312 is inserted to a shallow depth with active electrode areas 321 located at an activation site that is just below the surface and is physiologically coupled to wall tissue of the local chamber (e.g. right atrium). By locating the proximal active electrode areas 321 of the inner electrode 312 at the local chamber activation site, the inner electrode 312 senses contraction at a local sensing site within the conduction network of the local chamber (e.g. right atrium). When configured for unipolar sensing, the inner electrode 312 may have a single active electrode area 321. When configured for bipolar sensing, the inner electrode 312 may have two or more active electrode areas 321 that are physically spaced apart and electrically separated from one another. When two or more active electrode areas 321 are provided, they may be spaced slightly different or a common distance from the base 309.

The sensing circuit 322 is configured to perform bipolar sensing from pairs of active electrode areas to select electrical activity in the local chamber and in the adjacent chamber. Optionally, the sensing circuit 322 may perform unipolar sensing between a reference anode and a single active electrode area or a group of electrically common active electrode areas. The sensing circuit 322 measures a voltage potential difference between the voltage sensed at the first and second active electrode areas, or between a reference anode and the active sensing areas.

The inner and outer electrodes 310-312 may be formed as multiple cathode electrodes. The outer electrodes 310, 311 may be configured as a screw with a large pitch (e.g. length between adjacent turns), large diameter and may have a length that is relatively long, while the inner electrode 312 is configured as a screw with a small pitch, small diameter and shorter length.

The inner electrode 312 is shaped in a helix or screw and may be shorter or longer (e.g., extends a greater distance from the base) than the outer electrodes 310, 311. The electrodes 310-312 are fashioned to an appropriate length that permits it to drill a predetermined distance slightly into, or entirely through, the septum at the desired location. For example, the electrodes 310-312 may be provided with a desired length sufficient to extend through, or to a desired distance into, a septum region separating two chambers of the heart. For example, the outer electrodes 310, 311 may contact atrial wall tissue within the triangle of Koch, while the inner electrode 312 extends diagonally along the septum into the ventricular vestibule.

The inner electrode 312 may be formed as a single conductive wire or a bundle of conductive wires, where a distal portion of the wire is covered with insulation and the proximal portion is exposed to form the active electrode area 321. By covering the distal portion of the electrode 312 with insulation, this limits electrical conduction of the conductive wire to tissue surrounding the proximal portion at the active electrical areas 321, which senses electrical activity from the conductive network of the local chamber that is representative of physiologic behavior (e.g., conduction pattern) of the local chamber. Also, when delivering stimulus pulses, the active electrode areas 321 will deliver the pulses into the conductive network of the local chamber wall.

Optionally, a single reference anode electrode or multiple reference anode electrodes 318 may be provided for use when delivering a unipolar stimulus pulse. The anode electrode(s) 318 may be located along one or more sides of the shell 308, and/or on the top end 306 of the LIMD 300. Optionally, the entire shell 308 may be used as an anode electrode during unipolar sensing, unipolar pacing, cardioversion, defibrillation and the like.

The LIMD 300 includes a charge storage unit 324 and sensing circuit 322 within the housing 302. The sensing circuit 322 senses intrinsic or paced activity, while the change storage unit 324 stores high or low energy amounts to be delivered in one or more stimulus pulses.

The electrodes 310-312 may be used to deliver lower energy or high energy stimulus, such as pacing pulses, cardioverter pulse trains, defibrillation shocks and the like. The electrodes 310-312 may also be used to sense electrical activity, such as physiologic and pathologic behavior and events and provide sensed signals to the sensing circuit 322. The electrodes 310-312 are configured to be joined to an energy source, such as a charge storage unit 324. The electrodes 310-312 receive stimulus pulse(s) from the charge storage unit 324. The electrodes 310-312 may be the same or different size. The electrodes 310-312 are configured to deliver high or low energy stimulus pulses to the myocardium.

The LIMD 300 includes a controller 320, within the housing 302 308, to cause the charge storage unit 324 to deliver activation pulses through each of the electrodes 310-312 in a synchronous manner, based on information from the sensing circuit 322. The stimulus pulses are delivered synchronously to local and distal activation sites in the local and distal conduction networks such that stimulus pulses delivered at the distal activation site are timed to cause contraction of the adjacent chamber in a predetermined relation to contraction of the local chamber.

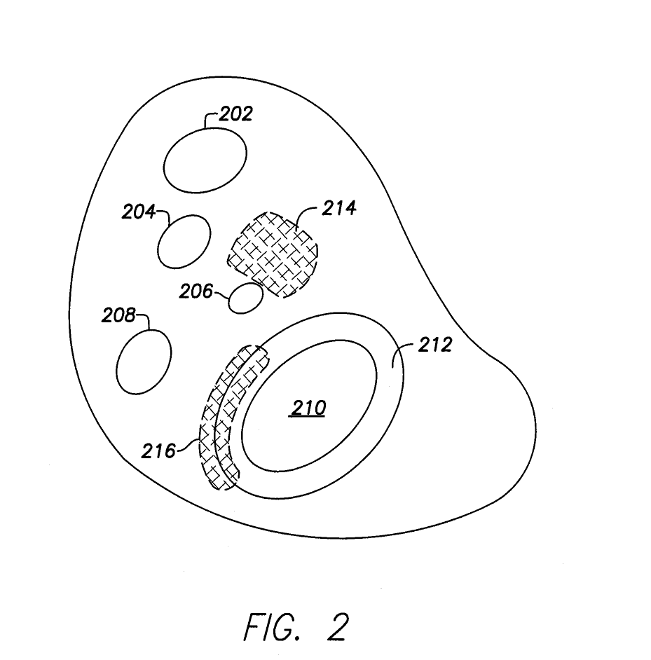

FIG. 2 illustrates a right anterior oblique view representing the interior surface of the right atrium wall. As shown in FIG. 2, the right atrium wall includes the superior vena cava (SVC) inlet 202, the fosa ovalis 204, coronary sinus 206, IVC 208, tricuspid valve 210 and tricuspid annulus 212 that surrounds the tricuspid valve 210. The LIMD 300 may be implanted in various locations within the RA. For example, the LIMD 300 may be implanted in region 214 which is located immediately adjacent the coronary sinus 206. Region 214 may be contained within the Triangle of Koch. For example, the LIMD 300 may be implanted in region 216 which may represent the ventricular vestibule in an area located adjacent the tricuspid valve 210 along a segment of the tricuspid annulus 212. Region 214 represents a local activation site in the local chamber wall at which contractions may be initiated when stimulus pulses are delivered to the surface tissue in the region 214 and electrodes deep in region 214 could stimulate adjacent tissue providing full DDD(R) sensing and pacing. Region 216, constitutes a distal activation site at which contractions may be initiated in the t ventricle when stimulus pulses are delivered in the region 216.

The controller 320 may operate the LIMD 300 in various modes, such as in select pacemaker modes, select cardiac resynchronization therapy modes, a cardioversion mode, a defibrillation mode and the like. For example, a typical pacing mode may include DDI, DDD or DDDR, DOO, VDD, VI, AAI and the like, where the first letter indicates the chamber(s) paced (e.g., A: Atrial pacing; V: Ventricular pacing; and D: Dual-chamber (atrial and ventricular) pacing). The second letter indicates the chamber in which electrical activity is sensed (e.g., A, V, or D). The code O is used when pacemaker discharge is not dependent on sensing electrical activity. The third letter refers to the response to a sensed electric signal (e.g., T: Triggering of pacing function; I: Inhibition of pacing function; D: Dual response (i.e., atrial sensed activity will inhibit atrial pacing but initiate (trigger) timing of an atrioventricular delay and subsequent ventricular pulse if no sensed ventricular activity occurs) and O: No response to an underlying electric signal (usually used for testing only).

As one example, the controller 320 may be configured with DDI, DOO, DDD or DDDR mode-capable and the LIMD 300 would be placed in the RA. The screw type electrodes 310, 311 are used to secure it in conductive branch region 214 (FIG. 2). Conductive branch region 214 is contained within the Triangle of Koch and is characterized by more ready activation of RA tissue compared to conductive branch region 216. When the LIMD 300 is secured in conductive branch region 216, it is possible to achieve Hisian/para-Hisian pacing from the RA and perform biventricular stimulation that is more consistent with normal physiology. It may be possible to also perform AV pacing from conductive branch region 216.

As one example, the conductive branch region 216 represents the adjacent chamber activation site within the ventricular vestibule. The inner electrode 312 delivers stimulus pulses to the ventricular vestibule to initiate activation in the right ventricle 37 of the heart. When the LIMD 300 is secured in the conductive branch region 216, the inner electrode 312 is located in a minor tissue portion that is non-responsive to the local events and local conduction occurring in the right atrium. The distal end 314 of the inner electrode 312 electrically engages the minor tissue portion that is responsive to non-local events and non-local conduction originating in another chamber.

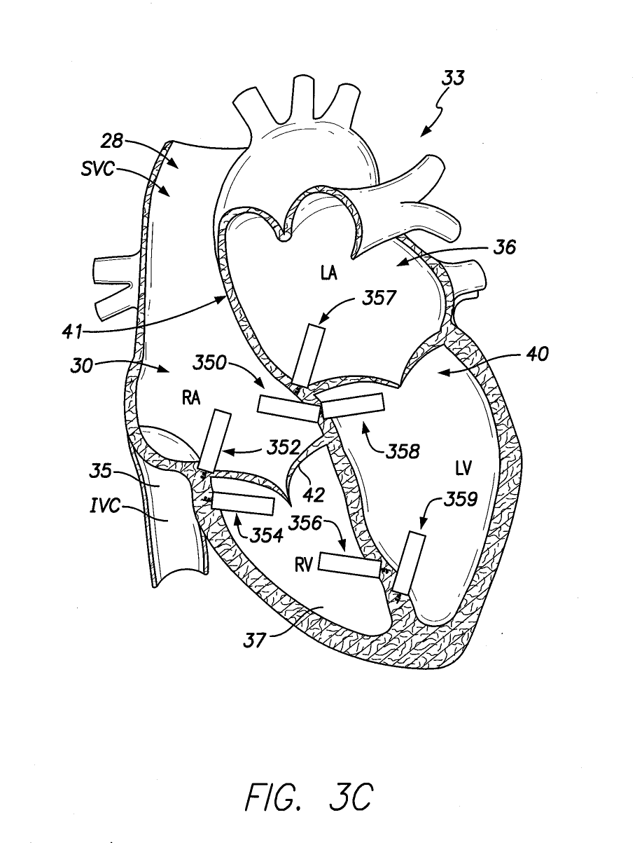

As shown in FIG. 4A, the sensing circuit 322 receives sensed signals from one or more of the electrodes 310-312. The sensing circuit 322 discriminates between sensed signals that originate in the near field and in the far field. For example, the electrodes 310-311 may be coupled to perform bipolar sensing of a voltage potential across small areas and thereby allow the sensing circuit 322 to discriminate between different sources of electrical signals.

The sensing circuit 322 measures, during bipolar sensing, a voltage potential difference between the voltages sensed at the active electrode areas 427 and 429. The sensing circuit 322 may compare the measured voltage potential difference to a threshold and only pass measured signals that exceed the threshold. The sensing circuit 322 reduces cross talk from far-field signals through the use of a threshold or some other filtering technique that analyzes the measured voltage potential.

In one embodiment, the electrode spacing between active electrode areas 317, 319 are limited or minimized in order to achieve a select type of sensing such as bipolar sensing which limits or minimizes sensing of far field signals. For example, during sensing, the electrode 310 may operate as an anode electrode and the electrode 311 may operate as a cathode electrode with a small separation (e.g. up to 2 mm) there between such that when far field signals (e.g., signals from the right atrium) reach the first and second electrodes these far field signals are sensed as a common mode signal with no or a very small potential difference between the electrodes. As one example, the active electrode areas 317, 319 may be circular and have a diameter of 0.4-0.6 mm, or up to 1.0 mm.

In another bipolar sensing configuration, the active electrode area 321 on electrode 312 may be split into a pair of electrically separate active electrode areas. The pair of active electrode areas may operate as an anode and as a cathode electrode with a small inter-electrode separation there between such that when far field signals (e.g., signals from the right ventricle) reach the first and second sensing regions these far field signals are sensed as a common mode signal with no or a very small potential difference between the sensing regions.

Optionally, an anode electrode 417 may be disposed along the lead body. The lead body may further carry a cardioverting-defibrillating electrode, which in one embodiment is in the form of an elongated coil wound about the outer surface of an insulating housing. Alternately, a cardioverting-defibrillating electrode may be in the form of a conductive polymer electrode. An inter-electrode spacing 460 separates the distal edge 462 of the anode electrode 417 from the proximal end of the pair 416. A spacing 466 separates the distal edge 462 of the anode electrode 417 from the distal electrode pair 418.

The housing 302 also includes a battery 326 that supplies power to the electronics and energy to the change storage unit 324.

FIG. 3C illustrates some of these possible configurations, namely at 350-356. The previous examples involve an LIMD implanted in the RA and capable of pacing the RV. Optionally, the LIMD may also be located in other locations. At 350, the LIMD is capable of HISian or para-HISian pacing to produce excitation of the RV and LV. When the LIMD is implanted at 352, the LIMD is able to provide RA/RV sensing and pacing from the RA. When the LIMD is implanted at 354, the LIMD is able to provide RA/RV sensing and pacing from the RV. When the LIMD is implanted at 356, the LIMD is able to provide RV/LV sensing and pacing from the RV. The LIMDs 357, 358 and 359 afford LA/RA pacing and sensing, LV/RA pacing and sensing, and LV/RV pacing and sensing, respectively. These implementations produce excitation of the RV and LV in a manner more consistent with normal physiological function.

FIG. 4A illustrates a side view of an end portion of an LIMD 400 implanted in a local chamber 401 of a heart. The LIMD 400 includes a housing 402 that is shaped in a tubular or cylindrical shape that extends along a longitudinal axis 405. The housing 402 is configured to be implanted entirely within a single local chamber 401 of the heart. The local chamber 401 has local wall tissue 403 that constitutes part of a conduction network of the local chamber 401. The LIMD 400 is positioned such that the base 404 is engaged against, and secured to, a local wall tissue 403. For example, the base 404 may be secured to a septum 420 that separates the local chamber 401 from an adjacent chamber 407. The adjacent chamber 407 having distal wall tissue 415. The distal wall tissue 415 is separated from the local wall tissue 403 by a septum depth 421. The distal wall tissue 415 constitutes part of a conduction network of the adjacent chamber 407.

An active fixation member 409 is coupled to the base 404 of the housing 402 and extends outward in a direction generally along the longitudinal axis 405 of the housing 402. The active fixation member 409 has a proximal segment 426 configured to extend slightly into the septum 420 to a local sensing site (generally denoted at 436). The local sensing site 436 may be at the surface of the local wall tissue 403. Optionally, the local sensing site 436 may include tissue below the surface of the local wall tissue 403. The local sensing site 436 generally includes any and all tissue within the conduction network of the local chamber 401 and that follows the depolarization pattern of the local chamber 401.

The active fixation member 409 has a distal segment 428 configured to extend at least partially through the septum 428 to a distal sensing site (generally denoted at 438). The distal sensing site 438 may be at the surface of the distal wall tissue 415. Optionally, the distal sensing site 438 may include tissue below the surface of the distal wall tissue 415. The distal sensing site 438 generally includes any and all tissue within the conduction network of the adjacent chamber 407.

The active fixation member 409 includes active electrode areas pairs 416 and 418 that are located within the proximal and distal segments 426 and 428, respectively. The electrode pair 416 includes active electrode areas 423 and 425 within the proximal segment 426, while the electrode pair 418 includes active electrode areas 427 and 429 in the distal segment 428. The electrode pairs 416 and 418 are coupled to the sensing circuit (e.g., 322 in FIG. 3A). The active electrode areas 427 and 429 are positioned such that the electrode pair 418 is electrically coupled to the conduction network of the adjacent chamber 407. The sensing circuit 322 detects, as near field signals, voltage potential differences originating within the conduction network of the adjacent chamber 407 that exceed the threshold. The sensing circuit 322 rejects, as far field signals, voltage potential differences originating within the conduction network of the local chamber 401 that fall below the threshold.

The local wall tissue 403 of the local chamber 401 is not part of the conductive network of a different adjacent chamber 407. Hence, the local wall tissue 403 of the local chamber 401 does not conduct or depolarize in response to an intrinsic or paced event that originates in the adjacent chamber 407. Instead, the local wall tissue 403 conveys electrical activity resulting from intrinsic or paced events in the adjacent chamber 407 as a far field signal.

The distal wall tissue 415 of the adjacent chamber 407 is not part of the conductive network of a different local chamber 401. Hence, the distal wall tissue 415 of the adjacent chamber 407 does not conduct or depolarize in response to an intrinsic or paced event that originates in the local chamber 401. Instead, the distal wall tissue 415 conveys electrical activity resulting from intrinsic or paced events in the local chamber 401 as a far field signal.

Disposed along the housing 402 is an anode electrode 417. The housing 402 may further carry a cardioverting-defibrillating electrode, which in one embodiment is in the form of a ring wound about the outer surface of an insulating housing 402. Alternately, a cardioverting-defibrillating electrode may be in the form of a conductive polymer electrode.

FIG. 4A also illustrates exemplary conduction patterns for local near field (NF) electrical activity 444, a distal NF electrical activity 444, far field (FF) electrical activity 440 originating in the local chamber 401, and FF electrical activity 442 originating in the adjacent chamber 407. It is understood, that the conduction patterns are merely a general illustration for discussion purposes only and do not correspond to a specific physiologic electrical behavior. The NF electrical activity 444 is representative of conduct or depolarize, along the conduction network of the local wall tissue 403 in response to an intrinsic or paced event that originates in the local chamber 401. As indicated by the arrows, the NF electrical activity 444 will propagate in one of two directions that extend generally in a common direction as the surface of the local wall tissue 403. For example, the NF electrical activity 444 may propagate in a direction from left to right generally in a common direction as the surface of the local wall tissue 403 in the example of FIG. 4A. Alternatively, the NF electrical activity 444 may propagate from right to left generally in a common direction as the surface of the local wall tissue 403. The NF electrical activity 444 induces a voltage differential that extends generally in the common direction as the NF electrical activity 444.

As indicated by the arrows, the NF electrical activity 446 will also propagate in one of two directions that extend generally in a common direction as the surface of the distal wall tissue 415. For example, the NF electrical activity 446 may propagate in a direction from left to right generally in a common direction as the surface of the distal wall tissue 415 in the example of FIG. 4A. Alternatively, the NF electrical activity 446 may propagate from right to left generally in a common direction as the surface of the distal wall tissue 415. The NF electrical activity 446 induces a voltage differential that extends generally in common direction as the NF electrical activity 446.

The FF electrical activity 440 and 442 does not generally propagate along a surface of a particular chamber. Instead, FF electrical activity 440 and 442 propagate away from a surface of a particular chamber. In the example of FIG. 4A, the FF electrical activity 440 propagates outward in a direction away from the surface of the local wall tissue 403. The FF electrical activity 440 is illustrated with a series of dashed lines that progressively move further apart from one another and that have dashed lines that progressively become shorter to illustrate that as the FF electrical activity 440 moves away from its source the FF electrical activity 440 spreads outward, becomes more decentralized or widely distributed and lowers in signal strength. The FF electrical activity 440 forms a low level voltage front that propagates generally in a direction across the septum depth 421 toward the surface of the distal wall tissue 415. Similarly, the FF electrical activity 442 propagates outward in a direction away from the surface of the distal wall tissue 415. As the FF electrical activity 442 moves away from its source, the FF electrical activity 442 spreads outward, becomes more decentralized or widely distributed and lowers in signal strength. The FF electrical activity 442 forms a low level voltage front that propagates generally in a direction across the septum depth 421 toward the surface of the local wall tissue 403.

The electrodes 423 and 425 are sized, shaped and spaced apart from one another in a manner that facilitates discrimination between near field and far field signals. The electrodes 423 and 425 are separated by an inter-electrode spacing 421 that is sufficient such that, as depolarization occurs along the local wall tissue and the NF electrical activity 444 moves across the electrodes 423 and 425, an associated voltage potential is created between the electrodes 423 and 425. This voltage potential is detected by the sensing circuit 322 as the near field signal. In the embodiments illustrated the orientation of the electrodes 423 and 425 relative to the direction of NF electrical activity 444 does not impact sensitivity and thus this orientation may vary.

Optionally, electrodes 423 and 425 may be oriented in one or more select orientations relative to the NF electrical activity 444. For example, the electrodes 423 and 425 may be oriented generally in-line with one another to be spatially separated along the direction of NF electrical activity 444.

Similarly, the electrodes 427 and 429 are sized, shaped and spaced apart from one another in a manner that facilitates discrimination between near field and far field signals. The active electrode areas 427 and 429 are spaced desired distance from a reference point on the active fixation member, such as a desired distance from the base 404. The electrodes 427 and 429 are separated by an inter-electrode spacing 431 that is sufficient such that as depolarization occurs along the local wall tissue 415 and the NF electrical activity 446 moves across the electrodes 427 and 429, an associated voltage potential is created between the electrodes 427 and 429. This voltage potential is detected by the sensing circuit 322 as the near field signal. In the embodiments illustrated the orientation of the electrodes 427 and 429 relative to the direction of NF electrical activity 446 does not impact sensitivity and thus this orientation may vary, although optionally, the electrodes 427 and 429 may be oriented in one or more select orientations relative to the NF electrical activity 446.

Turning now to the FF electrical activity 440 and 442, the electrodes 423 and 425 are separated by an inter-electrode spacing 421 that is small enough such that, as the FF electrical activity 442 traverses the electrodes 423 and 425, a common mode or very low voltage potential is created between the electrodes 423 and 425. This voltage potential is rejected by the sensing circuit 322 as a far field signal. In the embodiments illustrated, the orientation of the electrodes 423 and 425 relative to the direction of FF electrical activity 442 does not impact sensitivity and thus this orientation may vary. Optionally, the electrodes 423 and 425 may be oriented in one or more select orientations relative to the FF electrical activity 442. For example, the electrodes 423 and 425 may be oriented along an inter-electrode axis (extending parallel to the inter-electrode spacing 421) that is substantially perpendicular to the direction of FF electrical activity 442.

The electrodes 427 and 429 are separated by an inter-electrode spacing 431 that is small enough such that, as the FF electrical activity 440 traverses the electrodes 427 and 429, a common mode or very low voltage potential is created between the electrodes 427 and 429. This voltage potential is rejected by the sensing circuit 322 as a far field signal. In the embodiments illustrated, the orientation of the electrodes 427 and 429 relative to the direction of FF electrical activity 440 does not impact sensitivity and thus this orientation may vary. Optionally, the electrodes 427 and 429 may be oriented in one or more select orientations relative to the FF electrical activity 440. For example, the electrodes 427 and 429 may be oriented along an inter-electrode axis (that follows the arrow denoted by the inter-electrode spacing 431) that is substantially perpendicular to the direction of FF electrical activity 440. Optionally, the inter-electrode axis may extend in any direction that is non-parallel to the direction of the FF electrical activity 440.

In one embodiment, the inter-electrode spacing may be limited or minimized in order to achieve a select sensitivity level. The electrodes 427 and 429 perform bipolar sensing which limits or minimizes sensing of far field signals. By way of example, the electrode 427 may operate as an anode electrode and the electrode 429 may operate as a cathode electrode with a small separation there between such that when far field signals reach the electrodes 427 and 429 the far field signals are sensed as a common mode signal with no or a very small potential difference between the electrodes 427 and 429.

Disposed along the lead body is an anode electrode 417. The housing 402 may further carry a cardioverting-defibrillating electrode, which in one embodiment is in the form of an elongated coil wound about the outer surface of an insulating housing. Alternately, a cardioverting-defibrillating electrode may be in the form of a conductive polymer electrode. An inter-electrode spacing 460 separates the distal edge 462 of the anode electrode 417 from the proximal end of the pair 416. A spacing 466 separates the distal edge 462 of the anode, electrode 417 from the distal electrode pair 418.

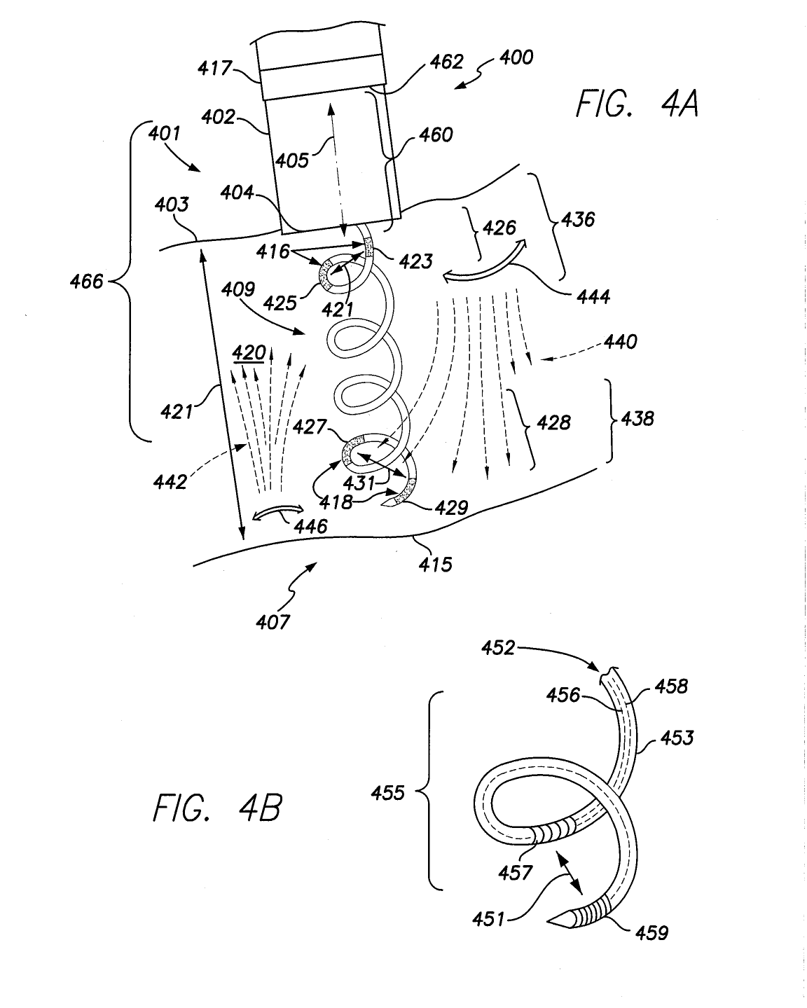

Various combinations of the electrodes illustrated in FIGS. 4 and 5 may be used to deliver stimulus pulses. During stimulation, one or more of the electrodes 423, 425, 427 and 429 may be electrically joined to one another (i.e., common), or may be maintained electrically separated. When one or more of the electrodes 423, 425, 427 and 429 are electrically joined to one another, a separate anode electrode may be provided on the housing 402.

The active fixation member 409 may be formed in accordance with several manners.

FIG. 4B illustrates a distal segment 455 of an active fixation member 452 formed in accordance with an embodiment. The distal segment 455 of the active fixation member 452 may be formed with a non-conductive helically shaped body 453 that has a lumen extending there through. The distal extremity of the active fixation member 452 includes active electrode areas 457 and 459 located upon separated turns or windings to provide an inter-electrode spacing 451 there between. Insulated conductive wires 456 and 458 extend along the lumen from the LIMD 300 to the corresponding electrode 457 and 459, respectively. The wires 456 and 458 form separate conductive paths between the sensing circuit 322 and the corresponding electrode 457 and 459.

FIG. 5 illustrates a side view of an end portion of an LIMD 500 implanted in a local chamber 501 of a heart. The LIMD 500 includes a housing 502 that is shaped in a tubular or cylindrical shape that extends along a longitudinal axis 505. The housing 502 is configured to be implanted entirely within a single local chamber 501 of the heart. The local chamber 501 has local wall tissue 503 that constitutes part of a conduction network of the local chamber 501. The LIMD 500 is positioned such that the base 504 is engaged against, and secured to, the local wall tissue 503. For example, the base 504 may be secured to a septum 520 that separates the local chamber 501 from an adjacent chamber 507. The adjacent chamber 507 having distal wall tissue 515. The distal wall tissue 515 is separated from the local wall tissue 503 by a septum depth 521. The distal wall tissue 515 constitutes part of a conduction network of the adjacent chamber 507.

An active fixation member 509 is coupled to the base 504 of the housing 502 and extends outward in a direction generally along the longitudinal axis 505 of the housing 502. The active fixation member 509 is helical in shape and winds around a needle-like structure or pin 511 that also extends from base 504. The base 504 engages the local wall tissue 503 at a local sensing site (generally denoted at 536). The pin 511 has a straight shaft that projects outward from a central area of the base 504. Optionally, the local sensing site 536 may include tissue below the surface of the local wall tissue 503.

The pin 511 has a distal segment 528 configured to extend at least partially through the septum to a distal sensing site (generally denoted at 538). The distal sensing site 538 may be at the surface of the distal wall tissue 515. Optionally, the distal sensing site 538 may include tissue below the surface of the distal wall tissue 515. The distal sensing site 538 generally includes any and all tissue within the conduction network 546 of the adjacent chamber 507. As shown in FIG. 5, the distal tip of the pin 511 may extend into the adjacent chamber 507. Optionally, the distal tip of the pin 511 may not extend into the adjacent chamber 507. Optionally, the distal tip of the active fixation member 509 may or may not extend into the adjacent chamber 507.

The base 504 includes an electrode pair 516 that is located at the local sensing site 536. The distal tip of the pin 511 includes an electrode pair 518 that are located at the distal sensing site 538. The electrode pair 516 includes active electrode areas 523 and 525 that are separated by an inter-electrode spacing 521 (e.g. 1 mm or up to 2 mm). The active electrode areas 523 and 525 may be circular bumps in shape with a diameter of 0.4 to 0.6 mm or up to 0.8 mm. The electrode pair 518 includes active electrode areas 527 and 529 that are separated by an inter-electrode spacing 531. The electrode pairs 516 and 518 are coupled to the sensing circuit (e.g., 322 in FIG. 3A). The electrodes 527 and 529 are positioned such that the electrode pair 518 is electrically coupled to the conduction network 546 of the adjacent chamber 507. The sensing circuit 322 detects at 527, 529, as near field signals 546, voltages originating within the conduction network of the adjacent chamber 507. The sensing circuit 322 rejects, as far field signals 540 sensed at 527, 529, voltages originating within the conduction network 544 of the local chamber 501. Similarly, near Field signals 544 sensed at 523, 525 are accepted, but signals 542 sensed at 523, 525 are rejected as far field signals

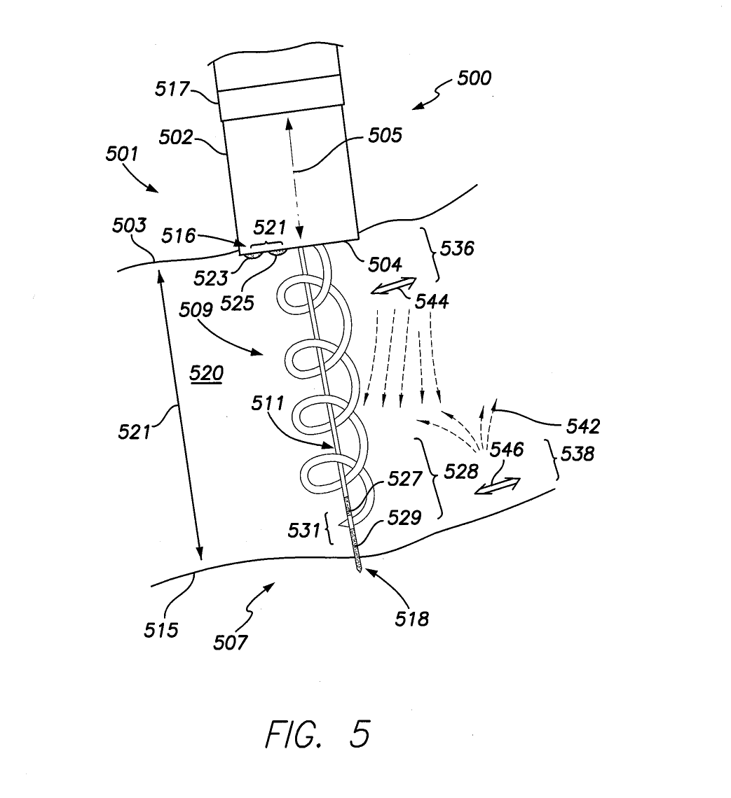

FIG. 6 illustrates a bottom plan view of a base formed in accordance with an embodiment. The base 604 includes an active fixation member 609 and/or pin 611, and a set 616 of three active electrode areas 623-625 as arranged in a triangular pattern. The active electrode areas 623-625 are separated by different inter-electrode spacing 631-633. The spacing 631-633 differ to afford multiple options for selecting a desired one of the spacing 631-633, based on which pair of active electrode areas 623 and 625 are chosen to be used for sensing. For example, active electrode areas 623-625 may be used, which have spacing 632 there between. Alternatively, active electrode areas 623 and 624 or 624 and 625 may be used.

Optionally, the active electrode areas 623-625 may have different surface areas and/or shapes, combinations of which may be chosen.

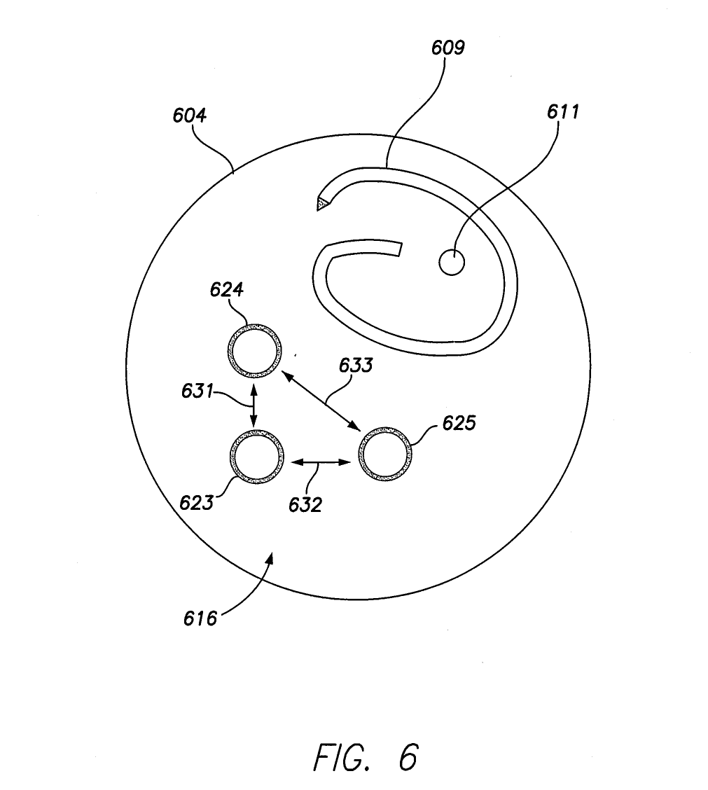

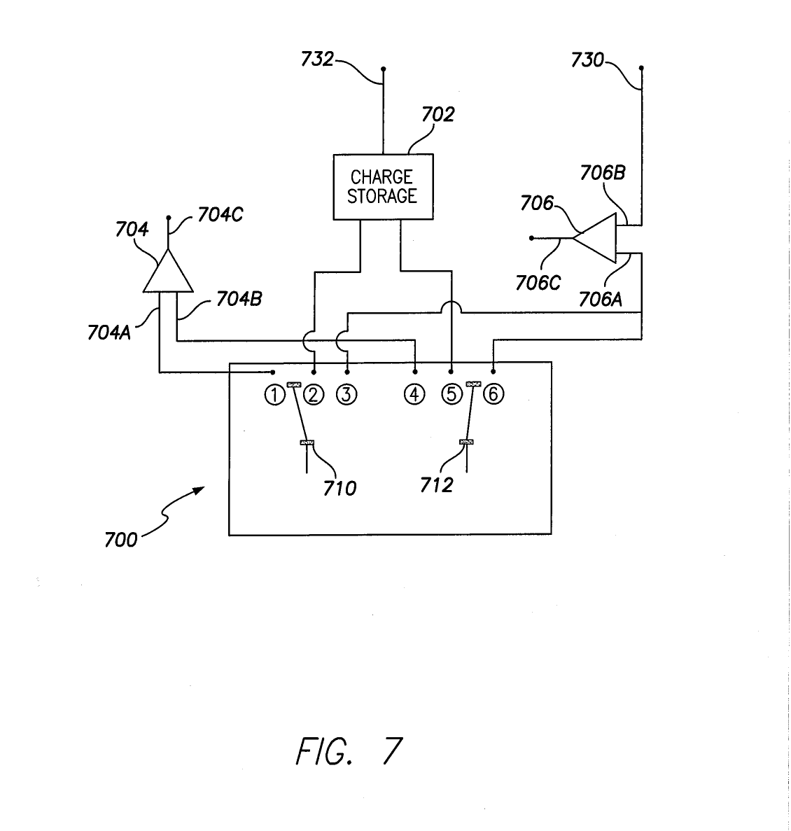

FIG. 7 illustrates a block diagram of an exemplary switching circuit that may be used in accordance with an embodiment of the present invention. The switching circuit 700 is coupled to the charge storage device 702 that is used to deliver stimulus pulses when delivering a therapy. The switching circuit 700 is connected to comparators 704 and 706 that form part of a sensing circuit (e.g. sensing circuit 322 in FIG. 3A or sensing circuit 844 in FIG. 8). The comparators 704 and 706 compare the voltage potentials at the inputs 704A, 704B and 706A and 706B, respectively. The comparators 704 and 706 output a corresponding differential signals at 704C and 706C to the programmable controller, such as controller 320 in FIG. 3A or controller 820 in FIG. 8. The switching circuit 700 includes inputs 710 and 712 that are configured to be connected to active electrode areas discussed in accordance with the embodiments herein. For example, the inputs 710 and 712 may represent the signals sensed at active electrode areas 427 and 429 (FIG. 4A), or the signals sensed at active electrode areas 423 and 425, or the signals sensed at active electrode areas 457 and 459 (FIG. 4B), or the signals sensed at active electrode areas 523 and 525 (FIG. 5), or active electrode areas 527 and 529, and the like. The switch 700 connects the inputs 710 and 712 to one of the corresponding contacts denoted at 1-6. For example, when the switch 700 connects the input 710 to the contact No. 1, the incoming signal is supplied to the input 704A for comparator 704. When the switch 700 connects input 712 to contact no. 6, the signal received on input 712 is supplied to the input 706A for comparator 706.

The comparator 706 also receives an input signal from a secondary electrode at 730, such as the reference anode electrodes 417, 517, 318 and the like. In accordance with one configuration, the switch 700 may change to a switch state to connect the inputs 710 and 712 to contacts no. 1 and 4 such that the comparator 704 will output a differential signal at 704C corresponding to the difference between the voltages at inputs 710 and 712.

In accordance with another switch state, the switch 700 may connect the input 710 and 712 to terminals no. 3 and 6 which are combined to render the electrodes connected to inputs 710 and 712 as a single common electrode, the signal for which is supplied to a single input 706A for comparator 706. This single input 706A is then compared to the signal received at 730 such that the comparator 706 outputs a differential signal at 706C corresponding to the difference between the voltage at 730 and the combined voltage received through contacts no. 3 and 6. The switch positions at contacts 1, 3, 4, and 6 correspond to sensor switch positions.

When the LIMD desires to deliver a stimulus pulse, the switch 700 changes the switch state such that the inputs 710 and 712 are then connected to contacts 2 and 5. Contacts 2 and 5 receive a stimulus pulse from the charged storage unit 702 in order that the charged storage unit 702 may deliver a stimulus pulse through switch 700 and input 710 and 712 to the correspondingly coupled electrodes. The charge storage device 702 also supplies a stimulus pulse to output terminal 732 which may be connected to the anode electrode, such as 318, 417 and 517 in FIGS. 3A, 4A and 5.

Optionally, the electrodes in the embodiments described herein may be formed as a separate conductive wire or a bundle of conductive wires, where a proximal portion of the wires are covered with insulation, while the distal tip is uncovered to be exposed. By covering the proximal portion of the wires with insulation, this limits electrical conduction of the conductive wire to tissue surrounding the distal. When implanted, the distal tip of the electrode is located far below the surface tissue of the chamber wall in which the LIMD is located. As a consequence, the distal tip of the electrode directly engages or is located proximate to the surface tissue of an adjacent chamber wall. Hence, the distal tip will sense electrical activity from the conductive network of the adjacent chamber that is representative of physiologic behavior (e.g., conduction pattern) of the adjacent chamber. Also, when delivering stimulus pulses, the distal tip will deliver the pulses into the conductive network of the adjacent chamber wall.

If dual-chamber pacing and sensing is achieved with a long helical fixation electrode covered proximally with insulation, it may be desirable to know when the helix has extended through the myocardium to the adjacent chamber. This may be determined using real-time impedance measurement between the helical tip electrode and another electrode. When the helical electrode is in pooled blood of any heart chamber, characteristic low impedance will be between it and any other electrode in the blood. As the helical electrode is screwed into the myocardium, impedance will rise. When the helix has been affixed sufficiently to break through the wall to the other chamber, impedance will drop. The changes in impedance may be used to know how far to screw in the helix, which portions of walls delineating heart chambers are an appropriate thickness for the helix, and whether any other spacer is needed to prevent the device from torqueing with the heart's mechanical motion.

If the fixation electrode is inserted so far that it penetrates into a chamber of the heart, a cap may be placed on the electrode. This cap may be accompanied by a lock on the other side that aids in fixation. Before disconnecting from the insertion tool, a pacing test provides an indication of the chamber paced and capture threshold. If the test shows that pacing is not occurring in the desired chamber or that thresholds are inappropriate, the tool may be used to remove the fixation and attempt to attach at another location.

For each attempt, the distance traversed by the lead's AV helix through the wall between the RA and RV between each turn of the screw may be closely controlled. Atrial and ventricular capture thresholds may be recorded with a pacing system analyzer (PSA) between each turn or at set degrees of rotation. The PSA may use the electrodes on the LIMD or may use electrodes on the exterior or outer end of the introducer to test for capture thresholds prior to affixing the LIMD in place. The distance between each turn may be 1 mm and all lead helical electrodes may be Parylene®-coated except for the most distal 1.5 mm pitch of the screws (thus ensuring that only tissue near the tip is stimulated). For example, a helical screw may traverse 6 mm into one chamber wall, while another helical screw may traverse 12 mm, 4 mm, and or 8 mm into another chamber wall before being able to contact and excite ventricular myocardium. In accordance with the foregoing, it is possible for an AV helical electrode on a lead to burrow from the RA and excite ventricular tissue. This allows a dual chamber mode-capable LIMD to have its main body located in the one chamber and pace and sense another chamber.

The term “distal” as used to describe wall tissue and activation sites, is used with respect to the local chamber.

FIG. 8 shows an exemplary LIMD 802 that is implanted into the patient as part of the implantable cardiac system 800. The LIMD 802 may be implemented as a pacemaker, equipped with both atrial and ventricular sensing and pacing circuitry for four chamber sensing and stimulation therapy (including both pacing and shock treatment). Optionally, the LIMD 802 may provide full-function cardiac resynchronization therapy. Alternatively, the LIMD 802 may be implemented with a reduced set of functions and components. For instance, the LIMD 802 may be implemented without ventricular sensing and pacing.

The LIMD 802 has a housing 800 to hold the electronic/computing components. The housing 800 (which is often referred to as the “can”, “case”, “encasing”, or “case electrode”) may be programmably selected to act as the return electrode for certain stimulus modes. Housing 800 further includes a connector (not shown) with a plurality of terminals 812, 804, 806, 808, and 810. The terminals may be connected to electrodes that are located in various locations within and about the heart. For example, the terminals may include: a terminal 812 to be coupled to an first electrode (e.g. a tip electrode) located in a first chamber; a terminal 804 to be coupled to a second electrode (e.g., tip electrode) located in a second chamber; a terminal 806 to be coupled to an electrode (e.g. ring) located in the first chamber; a terminal 808 to be coupled to an electrode located (e.g. ring electrode) in the second chamber; and a terminal 810 to be coupled to another electrode. The type and location of each electrode may vary. For example, the electrodes may include various combinations of ring, tip, coil and shocking electrodes and the like.

The LIMD 802 includes a programmable microcontroller 820 that controls various operations of the LIMD 802, including cardiac monitoring and stimulation therapy. Microcontroller 820 includes a microprocessor (or equivalent control circuitry), RAM and/or ROM memory, logic and timing circuitry, state machine circuitry, and I/O circuitry.

IMD 802 further includes a first chamber pulse generator 822 that generates stimulation pulses for delivery by one or more electrodes coupled thereto. The pulse generator 822 is controlled by the microcontroller 820 via control signal 824. The pulse generator 822 is coupled to the select electrode(s) via an electrode configuration switch 826, which includes multiple switches for connecting the desired electrodes to the appropriate I/O circuits, thereby facilitating electrode programmability. The switch 826 is controlled by a control signal 828 from the microcontroller 820.

In the example of FIG. 8, a single pulse generator 822 is illustrated. Optionally, the LIMD 802 may include multiple pulse generators, similar to pulse generator 822, where each pulse generator is coupled to one or more electrodes and controlled by the microcontroller 820 to deliver select stimulus pulse(s) to the corresponding one or more electrodes.

Microcontroller 820 is illustrated as including timing control circuitry 832 to control the timing of the stimulation pulses (e.g., pacing rate, atrio-ventricular (AV) delay, atrial interconduction (A-A) delay, or ventricular interconduction (V-V) delay, etc.). The timing control circuitry 832 may also be used for the timing of refractory periods, blanking intervals, noise detection windows, evoked response windows, alert intervals, marker channel timing, and so on. Microcontroller 820 also has an arrhythmia detector 834 for detecting arrhythmia conditions and a morphology detector 836. Although not shown, the microcontroller 820 may further include other dedicated circuitry and/or firmware/software components that assist in monitoring various conditions of the patient's heart and managing pacing therapies.

The LIMD 802 is further equipped with a communication modem (modulator/demodulator) 840 to enable wireless communication with external devices. In one implementation, the communication modem 840 uses high frequency modulation. As one example, the modem 840 transmits signals between a pair of electrodes. The signals are transmitted in a high frequency range of approximately 20-80 kHz, as such signals travel through the body tissue in fluids without stimulating the heart or being felt by the patient.

The communication modem 840 may be implemented in hardware as part of the microcontroller 820, or as software/firmware instructions programmed into and executed by the microcontroller 820. Alternatively, the modem 840 may reside separately from the microcontroller as a standalone component.

The LIMD 802 includes sensing circuitry 844 selectively coupled to one or more electrodes that perform sensing operations, through the switch 826 to detect the presence of cardiac activity in the right chambers of the heart.

The sensing circuit 844 is configured to perform bipolar sensing between one pair of electrodes and/or between multiple pairs of electrodes. The sensing circuit 844 detects NF electrical activity and rejects FF electrical activity.

The sensing circuitry 844 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit 802 to sense low amplitude signal characteristics of atrial fibrillation. Switch 826 determines the sensing polarity of the cardiac signal by selectively closing the appropriate switches. In this way, the clinician may program the sensing polarity independent of the stimulation polarity.

The output of the sensing circuitry 844 is connected to the microcontroller 820 which, in turn, triggers or inhibits the pulse generator 822 in response to the absence or presence of cardiac activity. The sensing circuitry 844 receives a control signal 846 from the microcontroller 820 for purposes of controlling the gain, threshold, polarization charge removal circuitry (not shown), and the timing of any blocking circuitry (not shown) coupled to the inputs of the sensing circuitry.

In the example of FIG. 8, a single sensing circuit 844 is illustrated. Optionally, the LIMD 802 may include multiple sensing circuit, similar to sensing circuit 844, where each sensing circuit is coupled to one or more electrodes and controlled by the microcontroller 820 to sense electrical activity detected at the corresponding one or more electrodes. The sensing circuit 844 may operate in a unipolar sensing configuration or in a bipolar sensing configuration.

The LIMD 802 further includes an analog-to-digital (A/D) data acquisition system (DAS) 850 coupled to one or more electrodes via the switch 826 to sample cardiac signals across any pair of desired electrodes. The data acquisition system 850 is configured to acquire intracardiac electrogram signals, convert the raw analog data into digital data, and store the digital data for later processing and/or telemetric transmission to an external device 854 (e.g., a programmer, local transceiver, or a diagnostic system analyzer). The data acquisition system 850 is controlled by a control signal 856 from the microcontroller 820.

The microcontroller 820 is coupled to a memory 860 by a suitable data/address bus 862. The programmable operating parameters used by the microcontroller 820 are stored in memory 860 and used to customize the operation of the LIMD 802 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 808 within each respective tier of therapy.

The operating parameters of the LIMD 802 may be non-invasively programmed into the memory 860 through a telemetry circuit 864 in telemetric communication via communication link 866 with the external device 854. The telemetry circuit 864 allows intracardiac electrograms and status information relating to the operation of the LIMD 802 (as contained in the microcontroller 820 or memory 860) to be sent to the external device 854 through the established communication link 866.

The LIMD 802 can further include magnet detection circuitry (not shown), coupled to the microcontroller 820, to detect when a magnet is placed over the unit. A magnet may be used by a clinician to perform various test functions of the unit 802 and/or to signal the microcontroller 820 that the external programmer 854 is in place to receive or transmit data to the microcontroller 820 through the telemetry circuits 864.

The LIMD 802 can further include one or more physiologic sensors 870. Such sensors are commonly referred to as “rate-responsive” sensors because they are typically used to adjust pacing stimulation rates according to the exercise state of the patient. However, the physiological sensor 870 may further be used to detect changes in cardiac output, changes in the physiological condition of the heart, or diurnal changes in activity (e.g., detecting sleep and wake states). Signals generated by the physiological sensors 870 are passed to the microcontroller 820 for analysis. The microcontroller 820 responds by adjusting the various pacing parameters (such as rate, AV Delay, V-V Delay, etc.) at which the atrial and ventricular pacing pulses are administered. While shown as being included within the unit 802, the physiologic sensor(s) 870 may be external to the unit 802, yet still be implanted within or carried by the patient. Examples of physiologic sensors might include sensors that, for example, sense respiration rate, pH of blood, ventricular gradient, temperature, activity, position/posture, minute ventilation (MV), and so forth.

A battery 872 provides operating power to all of the components in the LIMD 802. The battery 872 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). The battery 872 also desirably has a predictable discharge characteristic so that elective replacement time can be detected. As one example, the unit 802 employs lithium/silver vanadium oxide batteries.

The LIMD 802 further includes an impedance measuring circuit 874, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance; detecting when the device has been implanted; measuring stroke volume; and detecting the opening of heart valves; and so forth. The impedance measuring circuit 874 is coupled to the switch 826 so that any desired electrode may be used.

The microcontroller 820 further controls a shocking circuit 880 by way of a control signal 882. The shocking circuit 880 generates shocking pulses of low (e.g., up to 0.5 joules), moderate (e.g., 0.5-10 joules), or high energy (e.g., 811 to 40 joules), as controlled by the microcontroller 820. Such shocking pulses are applied to the patient's heart 808 through shocking electrodes. It is noted that the shock therapy circuitry is optional and may not be implemented in the LIMD, as the various slave pacing units described below will typically not be configured to deliver high voltage shock pulses. On the other hand, it should be recognized that the slave pacing unit can be used within a system that includes backup shock capabilities, and hence such shock therapy circuitry may be included in the LIMD.

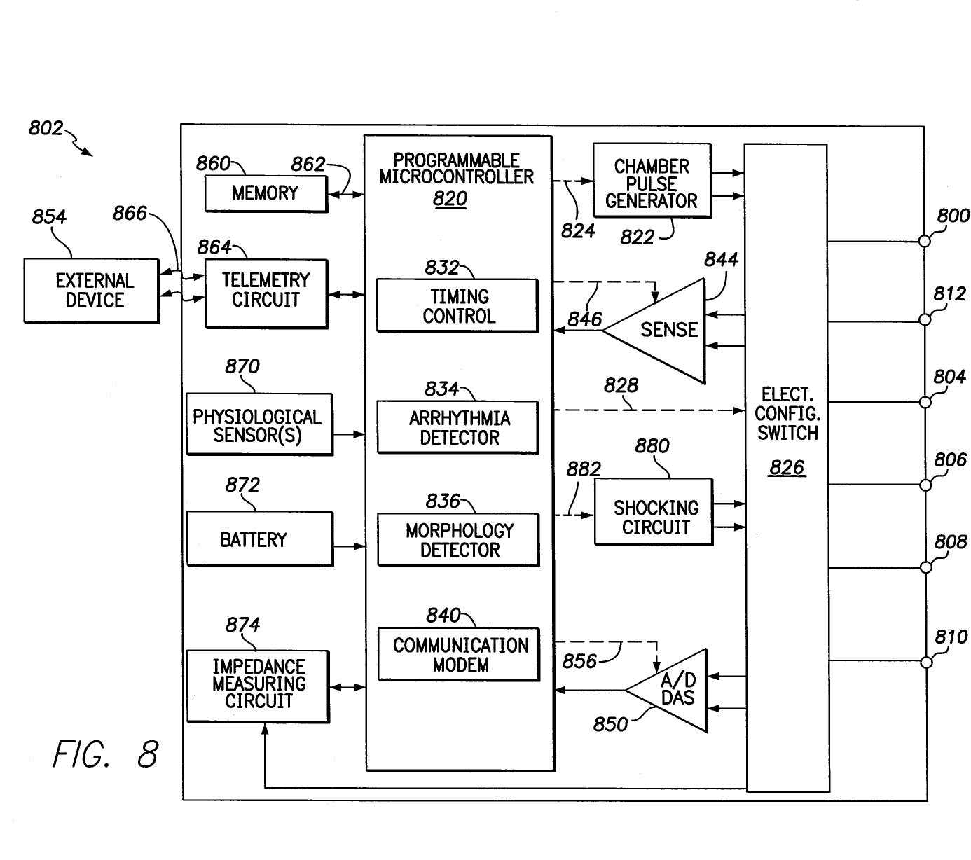

FIG. 9 illustrates a bottom plan view of an LIMD 900 formed in accordance with an alternative embodiment. The LIMD 900 comprises a proximal base 904, a distal top end (not shown), and a housing 902 extending between the proximal base 904 and the distal top end. The housing 902 is elongated and tubular in shape and extends along a longitudinal axis 909.

The base 904 includes inner and outer electrodes 910 and 912 securely affixed at base mounts 921 and 923 to the base 904. The inner and outer electrodes 910 and 912 projected outward from the base 904. For example, the outer electrode 912 is formed as a large semi-circular spike or large gauge wire that wrap about the inner electrode 910. The inner and outer electrodes 910 and 912 are physically and electrically separated from one another. The outer electrode 912 is positioned near the periphery of the base 904 and may expose a large portion of the conductive surface area thereof at the last 1-2 mm of the tip of the electrode 912. Optionally, the outer electrode 912 may have one or more active electrode areas that may be configured to operate as a cathode or an anode during sensing and/or during delivery of a stimulus pulse. The inner electrode 910 may extend outward along the longitudinal axis 909 and be shaped as a straight pin. The electrode 910 may have one or more active electrode area 914 located along the pin and/or at the distal end 916 thereof. The electrode 910 may be covered with insulation everywhere except the active electrode area 914. Optionally, a pin or needle 918 may extend beyond the active electrode area 914 to serve as a locating device. The electrode 910 may be configured to operate as a cathode during sensing and/or during delivery of a stimulus pulse. Optionally, needle 918 may be the active electrode area and area 914 may be insulated. Optionally, the inner electrode 912 may have a common diameter along the length thereof with a pointed needle tip.

The inner and outer electrodes 910 and 912 may be formed as a single conductive wires or bundles of conductive wires associated with each active electrode area, where none or a desired portion of the wire is covered with insulation, while a desired portion is exposed. By covering a portion of the electrodes 910 and 912 with insulation, this limits electrical conduction of the conductive wire to tissue surrounding the desired active electrode areas.

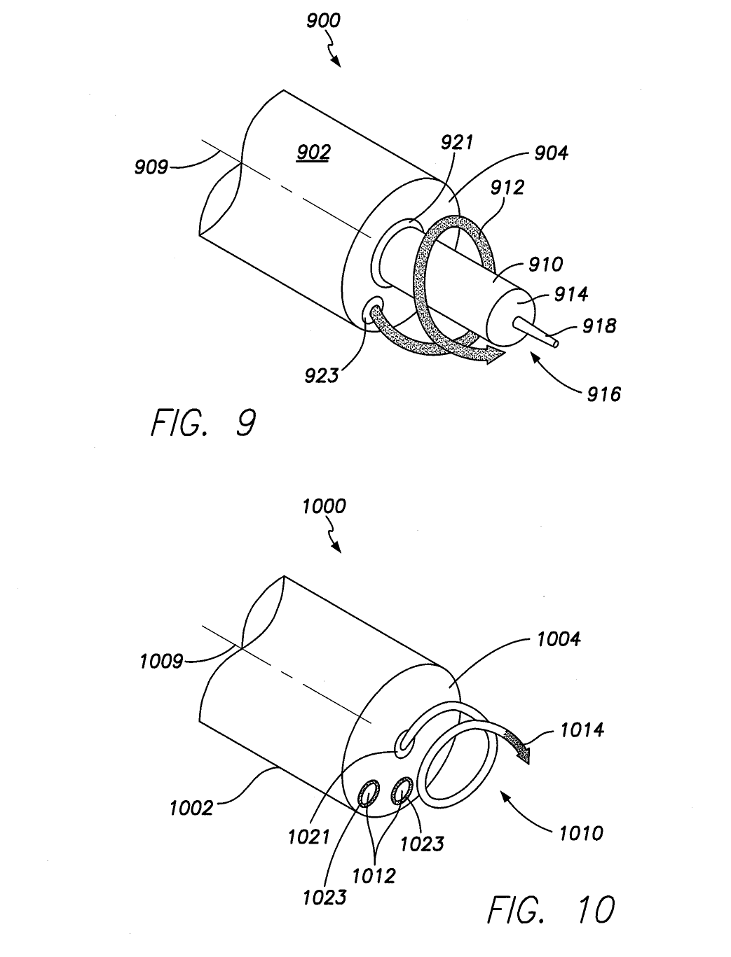

FIG. 10 illustrates a bottom plan view of an LIMD 1000 formed in accordance with an alternative embodiment. The LIMD 1000 comprises a proximal base 1004, a distal top end (not shown), and a housing 1002 extending between the proximal base 1004 and the distal top end. The base 1004 includes inner and outer electrodes 1010 and 1012 securely affixed at base mounts 1021 and 1023 to the base 1004. The inner and outer electrodes 1010 and 1012 project outward from the base 1004. For example, the outer electrodes 1012 may be formed as raised bump or surface electrodes that do not active affix to tissue. The inner and outer electrodes 1010 and 1012 are physically and electrically separated from one another. The outer electrodes 1012 are positioned near the periphery of the base 1004. The outer electrodes 1012 may be configured to operate one as an anode, both as cathodes, one as a cathode, both as anodes and the like during sensing and/or during delivery of a stimulus pulse. The inner electrode 1010 may extend outward along the longitudinal axis 1009 and be shaped as a helix or straight pin. The electrode 1010 may have one or more active electrode areas 1014 located at the distal end. The surface or bump type electrodes 1012 may be coupled to the conductive network of the local chamber (e.g. when positioned proximate the SA node or triangle of Koch and away from the ventricular vestibule). The electrode 1010 may be coupled to the conductive network of the adjacent chamber (e.g. when positioned proximate to the ventricular vestibule). Optionally, the electrodes 1012 may be coupled to the adjacent chamber when positioned within the ventricular vestibule. Optionally, the base mounts 921, 923, 1021 and 1023 may be formed with cavities in the bases 904 and 1004 and to surround the corresponding electrodes 910, 912, 1010, 1012. The cavities may represent circular indented pockets that receive a steroid or other biological agent that facilitates a desired behavior at the tissue wall that engages the electrodes 910, 912, 1010, 1012. For example, the steroid may encourage healing and discourage rejection of the electrode. As another example, the steroid may encourage the wall tissue to grow to the electrode and base. As another option, the steroid may reduce scarring when the wall tissue engages the electrode.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A leadless implantable medical device (LIMD), comprising:
a housing configured to be implanted entirely within a single local chamber of the heart, the local chamber having local wall tissue that constitutes part of a conduction network of the local chamber;
a controller within the housing to cause stimulus pulses to be delivered;
a sensing circuit to perform sensing;
an active fixation member coupled to the housing, the active fixation member configured to be secured to a septum that separates the local chamber from an adjacent chamber, the adjacent chamber having distal wall tissue, with respect to the local chamber, that constitutes part of a conduction network of the adjacent chamber; and
an electrode pair having first and second active electrode areas coupled to the sensing circuit, the first and second electrode areas positioned such that, when the LIMD is implanted, the electrode pair is electrically coupled to the conduction network of the adjacent chamber, the sensing circuit detecting, as near field signals, voltages originating within the conduction network of the adjacent chamber and sensed by the first and second active electrode areas, the sensing circuit rejecting, as far field signals, voltages originating within the conduction network of the local chamber and sensed by the first and second active electrode areas.

2. The LIMD of claim 1, wherein the sensing circuit and electrode pair are coupled to operate in a bipolar sensing configuration such that the sensing circuit measures a voltage potential difference between the first and second active electrode areas.

3. The LIMD of claim 1, wherein the active fixation member is helical in shape, the first and second active electrode areas being located on separate turns of the active fixation member and at a common distance from the base.

4. The LIMD of claim 1, wherein the electrode pair are provided on the active fixation member within a distal segment thereof, such that when the active fixation member is installed, the electrode pair are located at or near a surface of the distal wall tissue.

5. The LIMD of claim 1, further comprising a pin that extends from the base of the housing, the pin having a distal end with the active electrode areas provided at the distal end of the pin.

6. The LIMD of claim 1, further comprising a second electrode pair having third and fourth active electrode areas that are provided on the base of the housing, the third and fourth active electrode areas coupled to the sensing circuit and positioned such that the second electrode pair is electrically coupled to the conduction network of the local chamber, the sensing circuit detecting, as near field signals, voltages originating within the conduction network of the local chamber and sensed by the third and fourth active electrode areas, the sensing circuit rejecting, as far field signals, voltages originating within the conduction network of the adjacent chamber and sensed by the third and fourth active electrode areas.

7. The LIMD of claim 1, wherein the active fixation member includes a proximal segment configured to be located at a local sensing site, the LIMD further comprising a second electrode pair provided on the active fixation member in the proximal segment to be electrically coupled to the conduction network of the local chamber, the sensing circuit detecting, as near field signals, voltages originating within the conduction network of the local chamber, the sensing circuit rejecting, as far field signals, voltages originating within the conduction network of the adjacent chamber.

8. The LIMD of claim 1, wherein the active fixation member includes first and second electrode pairs that are located within proximal and distal segments of the active fixation member, respectively, the electrodes in the proximal segment being positioned to be electrically coupled to the conduction network of the local chamber.

9. The LIMD of claim 1, wherein the first and second electrodes are separated by an inter-electrode spacing such that as depolarization occurs along the distal wall tissue and near field electrical activity moves across the first and second electrodes, an associated voltage potential is created between the first and second electrodes, the voltage potential being detected by the sensing circuit as a near field signal.

10. The LIMD of claim 1, wherein the first and second electrodes are separated by an inter-electrode spacing such that as far field electrical activity traverses the first and second electrodes, a common mode signal is experienced between the first and second electrodes, the common mode signal being rejected by the sensing circuit.

11. A method for implanting a leadless implantable medical device (LIMD) the LIMD having a housing that includes a sensing circuit to perform sensing and an electrode pair having first and second active electrode areas coupled to the sensing circuit, the method comprising:
guiding the LIMD, utilizing an introducer, to an activation site that is located entirely within a single local chamber of the heart and proximate to tissue of interest, the local chamber having local wall tissue that constitutes part of a conduction network of the local chamber, an adjacent chamber having distal wall tissue, with respect to the local chamber, that constitutes part of a conduction network of the adjacent chamber; and
actively securing the LIMD to the tissue of interest;
positioning the electrode pair to engage wall tissue at a distal activation site within the conduction network of the adjacent chamber;
configuring the sensing circuit to detect, as near field signals, voltages originating within the conduction network of the adjacent chamber and sensed by the first and second active electrode areas; and
configuring the sensing circuit to reject, as far field signals, voltages originating within the conduction network of the local chamber and sensed by the first and second active electrode areas.

12. The method of claim 11, further comprising;
positioning at least a third electrode to engage wall tissue at a local activation site within the conduction network of the local chamber; and
configuring a controller within the housing to cause stimulus pulses to be delivered, in a synchronous manner, through the electrode pair and the third electrode to the distal and local activation sites, respectively, such that stimulus pulses delivered at the distal activation site are timed to cause contraction of the adjacent chamber in a predetermined relation to contraction of the local chamber.

13. The method of claim 11, further comprising configuring the sensing circuit and electrode pair to operate in a bipolar sensing configuration such that the sensing circuit measures a voltage potential difference between the first and second active electrode areas.

14. The method of claim 11, wherein the actively securing operation includes screwing an active fixation member, provided on the base of the housing of the LIMD, into the tissue of interest, the first and second active electrode areas being located on separate turns of the active fixation member.

15. The method of claim 11, wherein the electrode pair are located on the active fixation member within a distal segment thereof, such that when the active fixation member is installed, the electrode pair are located at or near a surface of the distal wall tissue.

16. The method of claim 11, further comprising securing a pusher tool to a proximal end of the LIMD within the introducer, utilizing the pusher tool to guide the LIMD into position, and utilizing the pusher tool to rotate the LIMD to actively secure a fixation member on the base of the LIMD to a septum.

17. The method of claim 11, further comprising providing a second electrode pair having third and fourth active electrode areas that are provided on a base of the housing, the third and fourth active electrode areas coupled to the sensing circuit and positioned such that the second electrode pair is electrically coupled to the conduction network of the local chamber, the sensing circuit detecting, as near field signals, voltages originating within the conduction network of the local chamber and sensed by the third and fourth active electrode areas, the sensing circuit rejecting, as far field signals, voltages originating within the conduction network of the adjacent chamber and sensed by the third and fourth active electrode areas.

18. The method of claim 11, wherein the active fixation member includes a proximal segment configured to be located at a local sensing site, the method further comprising providing a second electrode pair on the active fixation member in the proximal segment to be electrically coupled to the conduction network of the local chamber, the sensing circuit detecting, as near field signals, voltages originating within the conduction network of the local chamber, the sensing circuit rejecting, as far field signals, voltages originating within the conduction network of the adjacent chamber.

19. The method of claim 11, wherein the active fixation member includes first and second electrode pairs that are located within proximal and distal segments of the active fixation member, respectively, the active electrode areas in the proximal segment being positioned to be electrically coupled to the conduction network of the local chamber.

20. The method of claim 11, further comprising separating the first and second active electrode areas by an inter-electrode spacing such that as depolarization occurs along the distal wall tissue and near field electrical activity moves across the first and second active electrode areas, an associated voltage potential is created between the first and second electrodes, the voltage potential being detected by the sensing circuit as a near field signal.

21. The method of claim 11, further comprising separating the first and second electrodes by an inter-electrode spacing such that as far field electrical activity traverses the first and second active electrode areas, a common mode signal experienced between the first and second active electrode areas, the common mode signal being rejected by the sensing circuit.

22. The method of claim 11, further comprising performing dual chamber pacing and dual chamber sensing.

23. The method of claim 11, further comprising performing sensing only in a right ventricle and pacing in both a right atrium and the right ventricle.
